(19) United States
(12) Patent Application Publication
(10) Pub. No.: US 2011/0106126 A1
(43) Pub. Date: May 5, 2011

(54) INSERTER DEVICE INCLUDING ROTOR SUBASSEMBLY

(76) Inventors: Michael Love, Pleasanton, CA (US); Daniel H. Lee, Burlingame, CA (US); Heber Saravia, San Francisco, CA (US)

(21) Appl. No.: 12/873,133

(22) Filed: Aug. 31, 2010

Related U.S. Application Data
(60) Provisional application No. 61/238,646, filed on Aug. 31, 2009.

(51) Int. Cl. A61B 17/32

(52) U.S. Cl. 606/182

(57) ABSTRACT

An inserter subassembly including a rotor and drive member such that rotation of the rotor is translated to a linear motion including insertion and refraction paths.

### CROSS-REFERENCE TO RELATED APPLICATIONS

This application claims priority to U.S. Provisional Application No. 61/238,646, filed Aug. 31, 2009, which is incorporated by reference in its entirety herein for all purposes.

### FIELD OF THE INVENTION

The present invention relates generally to an inserter device, for example, to insert a medical device, e.g., an analyte sensor or an infusion set. More specifically, the present invention relates to an inserter device configured with a rotor subassembly.

### BACKGROUND OF THE INVENTION

Diabetes Mellitus is an incurable chronic disease in which the body does not produce or properly utilize insulin. Insulin is a hormone produced by the pancreas that regulates blood sugar (glucose). In particular, when blood sugar levels rise, e.g., after a meal, insulin lowers the blood sugar levels by facilitating blood glucose to move from the blood into the body cells. Thus, when the pancreas does not produce sufficient insulin (a condition known as Type 1 Diabetes) or does not properly utilize insulin (a condition known as Type II Diabetes), the blood glucose remains in the blood resulting in hyperglycemia or abnormally high blood sugar levels.

The vast and uncontrolled fluctuations in blood glucose levels in people suffering from diabetes cause long-term, serious complications. Some of these complications include blindness, kidney failure, and nerve damage. Additionally, it is known that diabetes is a factor in accelerating cardiovascular diseases such as atherosclerosis (hardening of the arteries), leading to stroke, coronary heart disease, and other diseases. Accordingly, one important and universal strategy in managing diabetes is to control blood glucose levels.

One way to manage blood glucose levels is testing and monitoring blood glucose levels by using conventional in vitro techniques, such as drawing blood samples, applying the blood to a test strip, and determining the blood glucose level using colorimetric, electrochemical, or photometric test meters. Another more recent technique for monitoring blood glucose levels is by using an in vivo continuous or automatic glucose monitoring system, such as for example, the FreeStyle Navigator® Continuous Glucose Monitoring System, manufactured by Abbott Diabetes Care, Inc. Unlike conventional blood glucose meters, continuous analyte monitoring systems employ an insertable or implantable sensor, which continuously detects and monitors blood glucose levels. Prior to each use of a new sensor, the user self implants at least a portion of the sensor under his skin. Typically, an inserter assembly is employed to insert the sensor in the body of the user. In this manner, an introducer sharp, while engaged to the sensor, pierces an opening into the skin of the user, releases the sensor and retracts from the body of the user. Accordingly, there exists a need for an easy-to-use, simple, insertion assembly which is reliable, minimizes pain, and is cost effective.

### SUMMARY

The invention provides an inserter subassembly, which includes a rotor and a driver member. The driver member can translate rotational motion of the rotor to a linear motion including a downward insertion direction and an upward retraction path. In some embodiments, the linear motion can be a reciprocating motion.

The inserter assembly can have improved reliability, e.g., improved sensor retention, smoothness of insertion and capture. For example, in some embodiments, the rotor can be coupled to a shuttle such that additional force or stored rotational energy exists for retraction to overcome the sensor retention means and release it from the introducer sharp. Additionally, the inserter assembly can be configured to cause less trauma during insertion, for example by exhibiting a smooth and guided motion into the skin, as opposed to a ballistic motion, and/or by spending less time in the skin during insertion.

In some embodiments, the inserter assembly includes a housing, a shuttle movably connected to the housing, an introducer sharp for piercing the skin of the user, a sensor for detecting and monitoring the analyte-of-interest, and a rotor for urging the introducer sharp and sensor towards an insertion direction, to an insertion point, and then towards a retraction direction and ultimately a retraction point. In some embodiments, the rotor is urged to rotate by a torsion spring. Additionally, when the inserter assembly is configured to transcutaneously insert an analyte sensor, the inserter assembly can be configured to attach to a mounting unit to define an insertion kit, which can be pre-loaded with the analyte sensor.

An introducer sharp, such as a metal sharp for piercing the skin, can be mounted to a surface of the shuttle. The introducer sharp can be mounted to the shuttle in a number of ways. For example, the introducer sharp and the shuttle can each be configured to have a snap-on engagement, as, for example, a shuttle including an extension or protrusion and a sharp including a recess or aperture. Alternatively, the introducer sharp can include an extension or protrusion and the shuttle can include a recess or aperture to define a snap-on engagement. Additionally or alternatively, the introducer sharp can be welded, glued, or otherwise mounted by heat shake. However, any known methods of securing the introducer sharp to the shuttle can be employed.

The introducer sharp can be configured to releasably hold the insertable sensor, which is configured to detect and monitor an analyte-of-interest in a biological sample, for example, glucose. The releasably-held insertable sensor may be held alternatively by features built onto the shuttle, housing, or other portion of the device.

In one embodiment, the shuttle is engaged to a rotor. The rotor has a pin extending axially and displaced radially from a surface which engages an elongate channel formed in the surface of the shuttle. In this manner, the engagement of the pin with the elongate channel can translate a single direction rotor rotation, e.g., clockwise or counterclockwise, to a linear motion, e.g., up and down. Thus, as the rotor can rotate along a rotational path, the forces from the pin applied to the channel can urge the shuttle in the linear component of the pin's movement. As the shuttle can be confined to a linear path, the resultant movement of the sharp along an downward and upward motion, toward an insertion and retraction direction. The linear path includes: the insertion direction, insertion point, retraction direction, and retraction point.

In an alternate embodiment, the rotor can be coupled to the shuttle portion of the device through a linkage. For example, an arm can control the movement of the shuttle in its linear movement. In another embodiment, a pivot located on the rotating element, connected through the linkage to a pivot point located on the shuttle can cause the shuttle through its movement.

In some embodiments, the bottom portion of the channel disposed or formed on the shuttle can be used to control the shuttle movement. Thus, rather than a channel, only a surface is needed as the interface between the rotor and the shuttle. The shuttle can be coupled to the housing by an additional spring element (other than that driving the rotor), towards the retraction position. As the rotor rotates along its rotational path, the pin forces on the shuttle surface urge the carrier downward. After the shuttle reaches its full depth, the additional housing spring element provides the retraction force on the shuttle. The upward motion of the shuttle can be limited by the continued rotation of the rotor pin.

In some embodiments, the rotor can be driven by a driver member, such as, but not limited to a spring, torsion drive spring, constant force spring, clock spring, rolled sheet metal, elastic member, or motor, and the like, which can be disposed between the housing and the rotor. In such embodiments, the rotor can include a catch feature, such as a projection, hole, slot, hex post, square post, to engage a catch member disposed on the spring or rolled sheet metal. In one embodiment, the rotor can be wound by a spline located centrally along the rotor axis. Engaging the spline and rotating the rotor will wind the spring. In this manner, the projection is capable of winding the spring or rolled sheet member when the rotor is wound. The unwinding of the spring or rolled sheet member drives the rotor along the rotational path, which translates into a linear path to insert an object into the user's body.

In some embodiments, the rotor can be driven by a rack and pinion type mechanism. The actual engagement of the rotor to the rack portion of the drive portion of the device could be through, for example, cables, friction, or by traditional toothed methods. The rack portion of the device can be constrained in movement to a singular direction. For example, the rack portion of the device can be in an upward position when the device is in an armed state. The user can manually push the rack downward via a handle attached to the rotor. Pressing the rack to a down position can rotate the rotor through a fixed rotation, for example. This rotation can drive the shuttle and sharp portion of the device through mechanisms, such as those described above. Alternatively, the rack could be preloaded with a spring element. Release of an actuation mechanism can release energy of the spring element and can drive the rack through its motion. Accordingly, this can translate to a fixed rotation of the rotor, which can then drive the shuttle and sharp portion of the device through its linear movement. The spring can include an extension spring or a compression spring. In yet other embodiments, the rotor can be driven by a piston crank, or rotor cam carrier housing. In some embodiments, a linkage, such as an arm can be linked to the rotor on a first end and the shuttle at the second end.

In some embodiments, the inserter assembly further includes an actuator for allowing the rotor to urge the shuttle and introducer sharp to the insertion point and then the retraction point. In some embodiments, the actuator includes a safety feature to impede the actuator until the safety feature is deactivated.

In some embodiments, the inserter can include a stopping member configured to slow down the rotor at the end of its motion before reaching a hard stop. For the purpose of illustration and not limitation, the rotor can include a brake. In this manner, a flexible member can be disposed on the rotor body to act as a friction brake against the rotor pin. In this regard, excess kinetic energy is dissipated to gradually slow the rotor motion prior to the end of motion. In another embodiment, the housing and/or the actuator can include one or more ribs configured to engage the rotor during rotation, such that the rotor is gradually slowed down and ultimately in a stop position.

In some embodiments, the inserter comprises a mounting unit releasably attached to the housing of the inserter and adapted to attach to a user's body at an insertion site. In this manner, the inserter is removed from the mounting unit after insertion of the sensor, and a transmitter, configured to transmit signals relating to the detected and monitored analyte-of-interest, is coupled to the mounting unit.

The inserter assembly can be used as a delivery device for various objects, including but not limited to a lancing device, infusion set, continuous glucose monitoring system sensor, including a transcutaneous sensor. The inserter can include disposable and/or reusable mechanisms.

These and other features, objects and advantages of the disclosed subject matter will become apparent to those persons skilled in the art upon reading the detailed description more fully described below.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a schematic view of the system in accordance with one embodiment of the disclosed subject matter;

FIGS. 2A and 2B are views, in partial cross section, of an electrochemical sensor in accordance with one embodiment of the disclosed subject matter;

FIG. 3 is a view, in partial cross section, of an electrochemical sensor in accordance with another embodiment of the disclosed subject matter;

FIG. 4 is an exploded view of one embodiment of an inserter assembly in accordance with the disclosed subject matter;

FIG. 5A-5E are schematic illustrations of the embodiment of the inserter assembly of FIG. 4;

FIG. 6A-6C are schematic illustrations of driver members that can be used in conjunction with a rotor in accordance with exemplary embodiments of the disclosed subject matter;

FIG. 7A is a schematic illustration of front and back views of an inserter subassembly including a rotor and rack and pinion at the pre-fire position in accordance with one embodiment of the disclosed subject matter;

FIG. 7B is a schematic illustration of front and back views of an inserter subassembly including a rotor and rack and pinion at the insertion position in accordance with one embodiment of the disclosed subject matter;

FIG. 7C is a schematic illustration of front and back views of an inserter subassembly including a rotor and rack and pinion at the retracted position in accordance with one embodiment of the disclosed subject matter;

FIGS. 8A-8C are schematic illustrations of the front views of an inserter subassembly in a pre-insertion, insertion, and retraction positions in accordance with embodiments of the disclosed subject matter;

FIGS. 9A-9C are schematic views of the back view of an inserter subassembly including a rotor, rack and pinion, and extension spring in a pre-insertion, insertion, and retracting positions in accordance with one embodiment of the disclosed subject matter;

FIGS. 10A-10C is a schematic view of the back view of an inserter subassembly including a rotor, rack and pinion, and compression spring in a pre-insertion, insertion, and retracting positions in accordance with one embodiment of the disclosed subject matter;

FIG. 11 is a schematic view of a rack and pinion having a cable in accordance with one embodiments of the disclosed subject matter;

FIG. 12 is a schematic view of a rack and pinion having a friction engagement in accordance with one embodiments of the disclosed subject matter;

FIG. 13 is a schematic view of a rack and pinion toothed profile gears in accordance with one embodiments of the disclosed subject matter

FIGS. 14A-14C are a schematic illustration of an inserter subassembly crank linkage in accordance with one embodiment of the disclosed subject matter;

FIGS. 15A-15C are a schematic illustration of an inserter subassembly crank linkage in accordance with another embodiment of the disclosed subject matter;

FIG. 16 is a schematic view of an inserter subassembly including a rotor and one or more cams configured to drive a shuttle in a linear motion;

FIG. 17 is a schematic illustration of an inserter subassembly including a rotor attached to an elastic member

FIG. 18 is a schematic illustration of a rotor including a brake in accordance with one embodiment of the disclosed subject matter.

FIG. 19 is a schematic illustration of an actuator including one or more ribs configured to gradually slow down motion of the rotor in accordance with one embodiments of the disclosed subject matter;

FIG. 20 is a perspective view of the one or more ribs of FIG. 19 in accordance with one embodiments of the disclosed subject matter; and

FIG. 21 is a schematic illustration of an actuator including one or more buttons configured to slow down or halt motion of the rotor in accordance with one embodiments of the disclosed subject matter.

FIG. 22 is a schematic illustration of an actuator including a dampening member configured to slow down or halt motion of the rotor in accordance with one embodiments of the disclosed subject matter.

### DETAILED DESCRIPTION OF THE EMBODIMENTS

A detailed description of the disclosure is provided herein. It should be understood, in connection with the following description, that the subject matter is not limited to particular embodiments described, as the particular embodiments of the subject matter may of course vary. It is also to be understood that the terminology used herein is for the purpose of describing particular embodiments only, and is not intended to be limiting, since the scope of the disclosed subject matter will be limited only by the appended claims.

Where a range of values is provided, it is understood that each intervening value between the upper and lower limit of that range and any other stated or intervening value in that stated range, is encompassed within the disclosed subject matter. Every range stated is also intended to specifically disclose each and every “subrange” of the stated range. That is, each and every range smaller than the outside range specified by the outside upper and outside lower limits given for a range, whose upper and lower limits are within the range from said outside lower limit to said outside upper limit (unless the context clearly dictates otherwise), is also to be understood as encompassed within the disclosed subject matter, subject to any specifically excluded range or limit within the stated range. Where a range is stated by specifying one or both of an upper and lower limit, ranges excluding either or both of those stated limits, or including one or both of them, are also encompassed within the disclosed subject matter, regardless of whether or not words such as “from”, “to”, “through”, or “including” are or are not used in describing the range.

Unless defined otherwise, all technical and scientific terms used herein have the same meaning as commonly understood by one of ordinary skill in the art to which this disclosed subject matter belongs. Although any methods and materials similar or equivalent to those described herein can also be used in the practice or testing of the present disclosed subject matter, this disclosure may specifically mention certain exemplary methods and materials.

All publications mentioned in this disclosure are, unless otherwise specified, incorporated herein by reference for all purposes, including without limitation to disclose and describe the methods and/or materials in connection with which the publications are cited.

The publications discussed herein are provided solely for their disclosure prior to the filing date of the present application. Nothing herein is to be construed as an admission that the present disclosed subject matter is not entitled to antedate such publication by virtue of prior invention. Further, the dates of publication provided may be different from the actual publication dates, which may need to be independently confirmed.

As used herein and in the appended claims, the singular forms “a”, “an”, and “the” include plural referents unless the context clearly dictates otherwise.

Nothing contained in the Abstract or the Summary should be understood as limiting the scope of the disclosure. The Abstract and the Summary are provided for bibliographic and convenience purposes and due to their formats and purposes should not be considered comprehensive.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosed subject matter. Any recited method can be carried out in the order of events recited, or in any other order which is logically possible. Reference to a singular item, includes the possibility that there are plural of the same item present. When two or more items (for example, elements or processes) are referenced by an alternative “or”, this indicates that either could be present separately or any combination of them could be present together except where the presence of one necessarily excludes the other or others.

Various exemplary embodiments of the analyte monitoring system and methods of the invention are described in further detail below. Although the invention is described primarily with respect to a glucose monitoring system, each aspect of the invention is not intended to be limited to the particular embodiment so described. Accordingly, it is to be understood that such description should not be construed to limit the scope of the invention, and it is to be understood that the analyte monitoring system can be configured to monitor a variety of analytes, as described below. Further, section headers, where provided, are merely for the convenience of the reader and are not to be taken as limiting the scope of the invention in any way, as it will be understood that certain elements and features of the invention have more than one function and that aspects of the invention and particular elements are described throughout the specification.

### A. Overview

The invention is generally directed to an inserter subassembly. The inserter subassembly can be configured to insert various devices into the body of a subject, such as for example, an analyte sensor, an infusion set, or a lancing device.

In one embodiment, the inserter subassembly can be a component of an inserter assembly configured to insert an analyte sensor for an analyte monitoring system, such as, for example, a continuous or semi-continuous glucose monitoring system.

Certain classes of analyte monitors are provided in small, lightweight, battery-powered and electronically-controlled systems. Such a system may be configured to detect signals indicative of in vivo analyte levels using an electrochemical sensor, and collect such signals, with or without processing the signal. In some embodiments, the portion of the system that performs this initial processing may be configured to provide the raw or initially processed data to another unit for further collection and/or processing. Such provision of data may be effected, for example, via a wired connection, such as an electrical, or via a wireless connection, such as an IR or RF connection.

Certain analyte monitoring systems for in vivo measurement employ a sensor that measures analyte levels in interstitial fluids under the surface of the subject's skin. These may be inserted partially through the skin (“transcutaneous”) or entirely under the skin (“subcutaneous”). A sensor in such a system may operate as an electrochemical cell. Such a sensor may use any of a variety of electrode configurations, such as a three-electrode configuration (e.g., with “working”, “reference” and “counter” electrodes), driven by a controlled potential (potentiostat) analog circuit, a two-electrode system configuration (e.g., with only working and counter electrodes), which may be self-biasing and/or self-powered, and/or other configurations. In some embodiments, the sensor may be positioned within a blood vessel.

In certain systems, the analyte sensor is in communication with a sensor control unit. As used in this disclosure, an on-body unit sometimes refers to such a combination of an analyte sensor with such a sensor control unit.

Certain embodiments are modular. The on-body unit may be separately provided as a physically distinct assembly, and configured to provide the analyte levels detected by the sensor over a communication link to a monitor unit, referred to in this disclosure as a “receiver unit” or “receiver device”, or in some contexts, depending on the usage, as a “display unit,” “handheld unit,” or “meter”. The monitor unit, in some embodiments, may include, e.g., a mobile telephone device, a personal digital assistant, other consumer electronic device such as MP3 device, camera, radio, etc., or other communication-enabled data processing device.

The monitor unit may perform data processing and/or analysis, etc. on the received analyte data to generate information pertaining to the monitored analyte levels. The monitor unit may incorporate a display screen, which can be used, for example, to display measured analyte levels, and/or audio component such as a speaker to audibly provide information to a user, and/or a vibration device to provide tactile feedback to a user. It is also useful for a user of an analyte monitor to be able to see trend indications (including the magnitude and direction of any ongoing trend), and such data may be displayed as well, either numerically, or by a visual indicator, such as an arrow that may vary in visual attributes, such as size, shape, color, animation, or direction. The receiver device may further incorporate an in vitro analyte test strip port and related electronics in order to be able to make discrete (e.g., blood glucose) measurements.

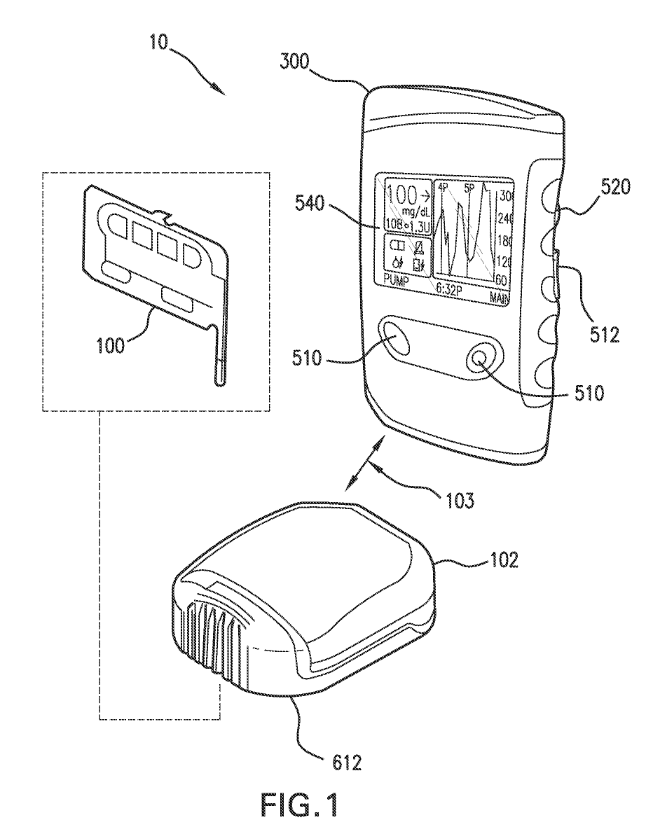

As illustrated in FIG. 1, the analyte monitoring system 10 may include a sensor 100, an on-body unit 102, a mount 612, and a monitor unit 300. Generally, the sensor 100 is configured to detect an analyte of interest and generate a signal relative to the level or concentration of the detected analyte in a biological sample of a user. The on-body unit 102 includes electronics configured to process the signal generated by the sensor 100 and may further include a transmitter, transceiver, or other communications electronics to provide the processed data to the monitor unit 300 via a communication link 103 between the transmitter and receiver. Further, the monitor unit 300 can include a display 540 for displaying or communicating information to the user of the analyte monitoring system 10 or the user's health care provider or another. In some embodiments, receiver 300 may also include buttons 510, 512 and/or scroll wheel 520 which allow a user to interact with a user interface located on receiver 300.

In the embodiment shown, on-body unit 102 and monitor unit 300 communicate via communications link 103 (in this embodiment, a wireless RF connection). Communication may occur, e.g., via RF communication, infrared communication, Bluetooth communication, Zigbee communication, 802.1x communication, or WiFi communication, etc., In some embodiments, the communication may include an RF frequency of 433 MHz, 13.56 MHz, or the like. In some embodiments, a secondary monitor unit may be provided. A data processing terminal may be provided for providing further processing or review of analyte data.

In certain embodiments, system 10 may be a continuous analyte monitor (e.g., a continuous glucose monitoring system or CGM), and accordingly operate in a mode in which the communications via communications link 103 has sufficient range to support a flow of data from on-body unit 102 to monitor unit 300. In some embodiments, the data flow in a CGM system is automatically provided by the on-body unit 102 to the monitor unit 300. For example, no user intervention may be required for the on-body unit 102 to send the data to the monitor unit 300. In some embodiments, the on-body unit 102 provides the signal relating to analyte level to the receiving unit 300 on a periodic basis. For example, the signal may be provided, e.g., automatically sent, on a fixed schedule, e.g., once every 250 ms, once a second, once a minute, etc. In some embodiments, the signal is provided to the monitor unit 300 upon the occurrence of an event, e.g., a hyperglycemic event or a hypoglycemic event, etc. In some embodiments, on-body unit 102 may further include local memory in which it may record, “logged data” or buffered data collected over a period of time and provide the some or all of the accumulated data to monitor unit 300 from time-to-time. Or, a separate data logging unit may be provided to acquire periodically received data from on-body unit 102. Data transmission may be one-way communication, e.g., the on-body unit 102 provides data to the monitor unit 300 without receiving signals from the monitor unit 300. In some embodiments, two-way communication is provided between the on-body unit 102 and the monitor unit 300.

In some embodiments, a signal is provided to the monitor unit 300 “on demand.” According to such embodiments, the monitor unit 300 requests a signal from the on-body unit 102, or the on-body unit 102 may be activated to send signal upon activation to do so. Accordingly, one or both of the on-body unit 102 and monitor unit 300 may include a switch activatable by a user or activated upon some other action or event, the activation of which causes analyte-related signal to be transferred from the on-body unit 102 to the monitor unit 300. For example, the monitor unit 300 is placed in close proximity with a transmitter device and initiates a data transfer, either over a wired connection, or wirelessly by various means, including, for example various RF-carried encodings and protocols and IR links.

In some embodiments, the signal relating to analyte level is instantaneously generated by the analyte sensor 100 upon receipt of the request, and provided to the monitor unit 300 as requested, and/or the signal relating to analyte level is periodically obtained, e.g., once every 250 ms, once a second, once a minute, etc. Upon receipt of the “on demand” request at the on-body unit 102, an analyte signal is provided to the monitor unit. In some cases, the signal provided to the monitor unit 300 is or at least includes the most recent analyte signal(s).

In further embodiments, additional data is provided to the monitor unit 300 “on demand.” For example, analyte trend data may be provided. Such trend data may include two or more analyte data points to indicate that analyte levels are rising, falling, or stable. Analyte trend data may include data from longer periods of time, such as, e.g., several minutes, several hours, several days, or several weeks.

Further details regarding on demand systems are disclosed in U.S. Pat. No. 7,620,438, U.S. Patent Publication Nos. 2009/0054749 A1, published Feb. 26, 2009; 2007/0149873 A1, published Jun. 28, 2007; 2008/0064937 A1, published Mar. 13, 2008; 2008/0071157 A1, published Mar. 20, 2008; 2008/0071158 A1, published Mar. 20, 2008; 2009/0281406 A1, published Nov. 12, 2009; 2008/0058625 A1, published Mar. 6, 2008; 2009/0294277 A1, published Dec. 3, 2009; 2008/0319295 A1, published Dec. 25, 2008; 2008/0319296 A1, published Dec. 25, 2008; 2009/0257911 A1, published Oct. 15, 2009, 2008/0179187 A1, published Jul. 31, 2008; 2007/0149875 A1, published Jun. 28, 2007; 2009/0018425 A1, published Jan. 15, 2009; and pending U.S. patent application Ser. Nos. 12/625,524, filed Nov. 24, 2009; 12/625,525, filed Nov. 24, 2009; 12/625,528, filed Nov. 24, 2009; 12/628,201, filed Nov. 30, 2009; 12/628,177, filed Nov. 30, 2009; 12/628,198, filed Nov. 30, 2009; 12/628,203, filed Nov. 30, 2009; 12/628,210, filed Nov. 30, 2009; 12/393,921, filed Feb. 27, 2009; 61/149,639, filed Feb. 3, 2009; 12/495,709, filed Jun. 30, 2009; 61/155,889, filed Feb. 26, 2009; 61/155,891, filed Feb. 26, 2009; 61/155,893, filed Feb. 26, 2009; 61/165,499, filed Mar. 31, 2009; 61/227,967, filed Jul. 23, 2009; 61/163,006, filed Mar. 23, 2009; 12/495,730, filed Jun. 30, 2009; 12/495,712, filed Jun. 30, 2009; 61/238,461, filed Aug. 31, 2009; 61/256,925, filed Oct. 30, 2009; 61/238,494, filed Aug. 31, 2009; 61/238,159, filed Aug. 29, 2009; 61/238,483, filed Aug. 31, 2009; 61/238,581, filed Aug. 31, 2009; 61/247,508, filed Sep. 30, 2009; 61/247,516, filed Sep. 30, 2009; 61/247,514, filed Sep. 30, 2009; 61/247,519, filed Sep. 30, 2009; 61/249,535, filed Oct. 7, 2009; 12/544,061, filed Aug. 19, 2009; 12/625,185, filed Nov. 24, 2009; 12/625,208, filed Nov. 24, 2009; 12/624,767, filed Nov. 24, 2009; 12/242,780, filed Sep. 30, 2008; 12/183,602, filed Jul. 31, 2008; 12/211,014, filed Sep. 15, 2008; and 12/114,359, filed May 2, 2008, each of which is incorporated by reference in its entirety herein.

### B. Sensor

The insertable sensor, in accordance with one embodiment of the invention, can be configured to detect and monitor an analyte of interest present in a biological sample of a user. The biological sample can be a biological fluid containing the analyte of interest, such as (but not limited to) interstitial fluid, blood, and urine. The analyte of interest can be one or more analytes including acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid stimulating hormone, and troponin. However, other suitable analytes can also be monitored, as would be known in the art. Furthermore, the analyte monitoring system can be configured to monitor the concentration of drugs, such as, for example, antibiotics (e.g., gentamicin, vancomycin, and the like), digitoxin, digoxin, theophylline, warfarin, and the like.

During use, the sensor is physically positioned in or on the body of a user whose analyte level is being monitored by an insertion device. The sensor can be configured to continuously sample the analyte level of the user and convert the sampled analyte level into a corresponding data signal for transmission by the transmitter. In some embodiments, the sensor is implantable into a subject's body for a period of time (e.g., three to seven days) to contact and monitor an analyte present in the biological fluid. Thus, a new sensor must be inserted typically every three to seven days. In one embodiment, the sensor can be a transcutaneous glucose sensor. Alternatively, the sensor can be a subcutaneous glucose sensor. The term “transcutaneous” as used herein refers to a sensor that is only partially inserted under one or more layers of the skin of the user, whereas the term “subcutaneous” refers to a sensor that is completely inserted under one or more layers of the skin of the user.

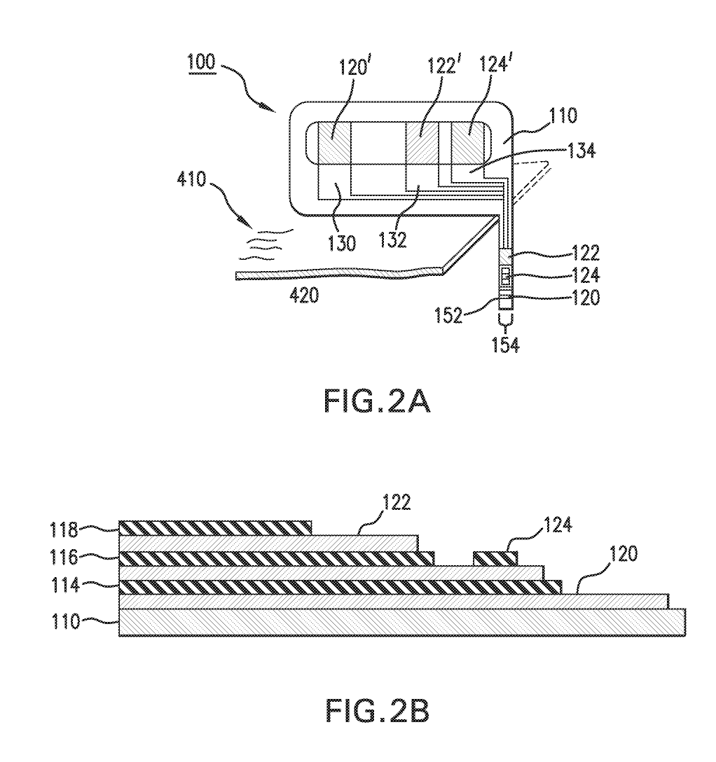

Generally, the sensor comprises a substrate, one or more electrodes, a sensing layer and a barrier layer, as described below and disclosed in U.S. Pat. Nos. 6,284,478 and 6,990,366, the disclosures of which are incorporated herein by reference. In one embodiment, as schematically illustrated in FIG. 2, the sensor 100 includes substrate 110. As the sensor is employed by insertion and/or implantation into a user's body for a period of days, in some embodiments, the substrate is formed from a relatively flexible material to improve comfort for the user and reduce damage to the surrounding tissue of the insertion site, e.g., by reducing relative movement of the sensor with respect to the surrounding tissue.

Suitable materials for a flexible substrate include, for example, non-conducting plastic or polymeric materials and other non-conducting, flexible, deformable materials. Suitable plastic or polymeric materials include thermoplastics such as polycarbonates, polyesters (e.g., Mylar® and polyethylene terephthalate (PET)), polyvinyl chloride (PVC), polyurethanes, polyethers, polyamides, polyimides, or copolymers of these thermoplastics, such as PETG (glycol-modified polyethylene terephthalate). In other embodiments, the sensor includes a relatively rigid substrate. Suitable examples of rigid materials that may be used to form the substrate include poorly conducting ceramics, such as aluminum oxide and silicon dioxide. Further, the substrate can be formed from an insulating material. Suitable insulating materials include polyurethane, teflon (fluorinated polymers), polyethyleneterephthalate (PET, Dacron) or polyimide.

As further depicted in FIG. 2A, substrate 110 can include a distal end 152 and a proximal end having different widths. In some embodiments, the proximal end of the sensor remains above the skin surface 410.

In such embodiments, the distal end 152 of the substrate 110 may have a relatively narrow width 154. Moreover, sensors intended to be subcutaneously or transcutaneously positioned into the tissue of a user's body at 420 can be configured to have narrow distal end or distal point to facilitate the insertion of the sensor. For example, for insertable sensors designed for continuous or periodic monitoring of the analyte during normal activities of the patient, a distal end 152 of the sensor 100 which is to be implanted into the user has a width of 2 mm or less, preferably 1 mm or less, and more preferably 0.5 mm or less.

A plurality of electrodes can be disposed near the distal end 152 of sensor 100. The electrodes include working electrode 120, counter electrode 122 and reference electrode 124. Other embodiments, however, can include a greater or fewer number of electrodes.

Each of the electrodes is formed from conductive material, for example, a non-corroding metal or carbon wire. Suitable conductive materials include, for example, vitreous carbon, graphite, silver, silver-chloride, platinum, palladium, or gold. The conductive material can be applied to the substrate by various techniques including laser ablation, printing, etching, silk-screening, and photolithography. In one embodiment, each of the electrodes are formed from gold by a laser ablation technique. As further illustrated, the sensor 100 includes conductive traces 130, 132, and 134 extending from electrodes 120, 122, and 124 to corresponding, respective contacts 120′, 122′, 124′ to define the sensor electronic circuitry. In one embodiment, an insulating substrate 114, 116, 118 (e.g., dielectric material) and electrodes 120, 122, 124 are arranged in a stacked orientation (i.e., insulating substrate disposed between electrodes), as shown in FIG. 2B. Alternatively, the electrodes can be arranged in a side by side orientation (not shown), as described in U.S. Pat. No. 6,175,752, the disclosure of which is incorporated herein by reference.

As schematically illustrated in FIG. 3, sensor 100 includes a sensing material 140. Sensing material 140 includes one or more components designed to facilitate the electrolysis of the analyte of interest. The components, for example, may be immobilized on the working electrode 120. Alternatively, the components of the sensing layer 140 may be immobilized within or between one or more membranes or films disposed over the working electrode 120 or the components may be immobilized in a polymeric or sol-gel matrix. Examples of immobilized sensing layers are described in U.S. Pat. Nos. 5,262,035, 5,264,104, 5,264,105, 5,320,725, 5,593,852, and 5,665,222, each of which is incorporated herein by reference.

The sensing layer components can include, for example, a catalyst to catalyze a reaction of the analyte at the working electrode 120, or an electron transfer agent to indirectly or directly transfer electrons between the analyte and the working electrode 120, or both. The catalyst is capable of catalyzing a reaction of the analyte. The catalyst may also, in some embodiments, act as an electron transfer agent. One example of a suitable catalyst is an enzyme which catalyzes a reaction of the analyte. For example, a catalyst, such as a glucose oxidase, glucose dehydrogenase (e.g., pyrroloquinoline quinone glucose dehydrogenase (PQQ)), or oligosaccharide dehydrogenase, may be used when the analyte is glucose. A lactate oxidase or lactate dehydrogenase may be used when the analyte is lactate. Laccase may be used when the analyte is oxygen or when oxygen is generated or consumed in response to a reaction of the analyte.

Preferably, the catalyst is non-leachably disposed on the sensor, whether the catalyst is part of a solid sensing layer in the sensor or solvated in a fluid within the sensing layer. More preferably, the catalyst is immobilized within the sensor (e.g., on the electrode and/or within or between a membrane or film) to prevent unwanted leaching of the catalyst away from the working electrode 120 and into the user. This may be accomplished, for example, by attaching the catalyst to a polymer, cross linking the catalyst with another electron transfer agent (which, as described above, can be polymeric), and/or providing one or more barrier membranes or films with pore sizes smaller than the catalyst.

In many embodiments, the sensing layer 140 contains one or more electron transfer agents in contact with the conductive material of the working electrode 120. In particular, for an implantable sensor, preferably, at least 90%, more preferably, at least 95%, and most preferably, at least 99%, of the electron transfer agent remains disposed on the sensor after immersion in the body fluid at 37° C. for 24 hours, and, more preferably, for 72 hours. Like the catalyst, the electron transfer agent can be immobilized on the working electrode. Suitable immobilization techniques include, for example, a polymeric or sol-gel immobilization technique. Alternatively, the electron transfer agent may be chemically (e.g., ionically, covalently, or coordinatively) bound to the working electrode, either directly or indirectly through another molecule, such as a polymer, that is in turn bound to the working electrode. The electron transfer agent mediates the transfer of electrons to electrooxidize or electroreduce an analyte and thereby permits a current flow between the working electrode 120 and the counter electrode 124 via the analyte. The mediation of the electron transfer agent facilitates the electrochemical analysis of analytes which are not suited for direct electrochemical reaction on an electrode. Useful electron transfer agents and methods for producing them are described in U.S. Pat. Nos. 5,264,104; 5,356,786; 5,262,035, 5,320,725, 6,990,366, each of which is incorporated herein by reference. Although any organic or organometallic redox species can be bound to a polymer and used as an electron transfer agent, the preferred redox species is a transition metal compound or complex. The preferred transition metal compounds or complexes include osmium, ruthenium, iron, and cobalt compounds or complexes. The most preferred are osmium compounds and complexes. It will be recognized that many of the redox species described below may also be used, typically without a polymeric component, as electron transfer agents in a carrier fluid or in a sensing layer of a sensor where leaching of the electron transfer agent is acceptable.

One type of non-releasable polymeric electron transfer agent contains a redox species covalently bound in a polymeric composition. An example of this type of mediator is poly(vinylferrocene). Another type of non-releasable electron transfer agent contains an ionically-bound redox species. Typically, this type of mediator includes a charged polymer coupled to an oppositely charged redox species. Examples of this type of mediator include a negatively charged polymer such as Nafion® (DuPont) coupled to a positively charged redox species such as an osmium or ruthenium polypyridyl cation. Another example of an ionically-bound mediator is a positively charged polymer such as quaternized poly(4-vinyl pyridine) or poly(1-vinyl imidazole) coupled to a negatively charged redox species such as ferricyanide or ferrocyanide. The preferred ionically-bound redox species is a highly charged redox species bound within an oppositely charged redox polymer.

In another embodiment of the invention, suitable non-releasable electron transfer agents include a redox species coordinatively bound to a polymer. For example, the mediator may be formed by coordination of an osmium or cobalt 2,2′-bipyridyl complex to poly(1-vinyl imidazole) or poly(4-vinyl pyridine). The preferred electron transfer agents are osmium transition metal complexes with one or more ligands, each ligand having a nitrogen-containing heterocycle such as 2,2′-bipyridine, 1,10-phenanthroline, or derivatives thereof. Furthermore, the preferred electron transfer agents also have one or more ligands covalently bound in a polymer, each ligand having at least one nitrogen-containing heterocycle, such as pyridine, imidazole, or derivatives thereof. These preferred electron transfer agents exchange electrons rapidly between each other and the working electrode 120 so that the complex can be rapidly oxidized and reduced.

One example of a particularly useful electron transfer agent includes (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same. Preferred derivatives of 2,2′-bipyridine for complexation with the osmium cation are 4,4′-dimethyl-2,2′-bipyridine and mono-, di-, and polyalkoxy-2,2′-bipyridines, such as 4,4′-dimethoxy-2,2′-bipyridine. Preferred derivatives of 1,10-phenanthroline for complexation with the osmium cation are 4,7-dimethyl-1,10-phenanthroline and mono, di-, and polyalkoxy-1,10-phenanthrolines, such as 4,7-dimethoxy-1,10-phenanthroline. Preferred polymers for complexation with the osmium cation include polymers and copolymers of poly(1-vinyl imidazole) (referred to as “PVI”) and poly(4-vinyl pyridine) (referred to as “PVP”). Suitable copolymer substituents of poly(1-vinyl imidazole) include acrylonitrile, acrylamide, and substituted or quaternized N-vinyl imidazole. Most preferred are electron transfer agents with osmium complexed to a polymer or copolymer of poly(1-vinyl imidazole).

To electrolyze the analyte, a potential (versus a reference potential) is applied across the working and counter electrodes 120,122. When a potential is applied between the working electrode and the counter electrode, an electrical current will flow. The current is a result of the electrolysis of the analyte or a second compound whose level is affected by the analyte. In one embodiment, the electrochemical reaction occurs via an electron transfer agent and the optional catalyst. Many analytes are oxidized (or reduced) to products by an electron transfer agent species in the presence of an appropriate catalyst (e.g., an enzyme). The electron transfer agent is then oxidized (or reduced) at the electrode. Electrons are collected by (or removed from) the electrode and the resulting current is measured. As an example, an electrochemical sensor may be based on the reaction of a glucose molecule with two non-leachable ferricyanide anions in the presence of glucose oxidase to produce two non-leachable ferrocyanide anions, two hydrogen ions, and gluconolactone. The amount of glucose present is assayed by electrooxidizing the non-leachable ferrocyanide anions to non-leachable ferricyanide anions and measuring the current. Changes in the concentration of the reactant compound, as indicated by the signal at the working electrode, correspond inversely to changes in the analyte (i.e., as the level of analyte increase then the level of reactant compound and the signal at the electrode decreases.).

The sensing layer 140 may be formed as a solid composition of the desired components (e.g., an electron transfer agent and/or a catalyst). However, in other embodiments, one or more of the components of the sensing layer 140 may be solvated, dispersed, or suspended in a fluid within the sensing layer 140, instead of forming a solid composition. The fluid may be provided with the sensor 100 or may be absorbed by the sensor 100 from the analyte-containing fluid. Preferably, the components which are solvated, dispersed, or suspended in this type of sensing layer 140 are non-leachable from the sensing layer.

Non-leachability may be accomplished, for example, by providing barriers (e.g., the electrode, substrate, membranes, and/or films) around the sensing layer which prevent the leaching of the components of the sensing layer 140. One example of such a barrier layer is a microporous membrane or film which allows diffusion of the analyte into the sensing layer 140 such that contact is made with the components of the sensing layer 140, but reduces or eliminates the diffusion of the sensing layer components (e.g., a electron transfer agent and/or a catalyst) out of the sensing layer 140.

A variety of different sensing layer configurations can be used. In one embodiment, the sensing layer 140 is deposited on at least a portion of the conductive material of a working electrode 120, as illustrated in FIG. 3. The sensing layer 140 may extend beyond the conductive material of the working electrode 120. For example, in some embodiments, the sensing layer 140 may also extend over the counter electrode 122 or reference electrode 124 without degrading the performance of the sensor. In other embodiments, the sensing layer can extend over the entire sensor substrate or tail of the sensor substrate, as described in U.S. Patent Application No. 61/165,499, the disclosure of which is incorporated by reference.

In some embodiments, as depicted in FIG. 3, the sensor includes a barrier layer 150 to act as a diffusion-limiting barrier to reduce the rate of mass transport of the analyte into the region around the working electrode 120. A steady state concentration of the analyte in the proximity of the working electrode (which is proportional to the concentration of the analyte in the body or sample fluid) is established by limiting the diffusion of the analyte. This extends the upper range of analyte concentrations that can still be accurately measured and may also expand the range in which the current increases approximately linearly with the level of the analyte.

It is preferred that the permeability of the analyte through the barrier layer 150 vary little or not at all with temperature, so as to reduce or eliminate the variation of current with temperature. For this reason, it is preferred that in the biologically relevant temperature range from about 25° C. to about 45° C., and most importantly from 30° C. to 40° C., neither the size of the pores in the film nor its hydration or swelling change excessively. Preferably, the barrier layer is made using a film that absorbs less than 5 wt. % of fluid over 24 hours. For implantable sensors, it is preferable that the barrier layer is made using a film that absorbs less than 5 wt. % of fluid over 24 hours at 37° C. Particularly useful materials for the barrier layer 150 include membranes that do not swell in the analyte-containing fluid that the sensor tests. Suitable membranes include 3 to 20,000 nm diameter pores. Membranes having 5 to 500 nm diameter pores with well-defined, uniform pore sizes and high aspect ratios are preferred. In one embodiment, the aspect ratio of the pores is preferably two or greater and more preferably five or greater. It is preferred that the permeability of the barrier layer membrane changes no more than 4%, preferably, no more than 3%, and, more preferably, no more than 2%, per ° C. in the range from 30° C. to 40° C. when the membranes resides in the subcutaneous interstitial fluid.

In some embodiments of the invention, the barrier layer 150 can also limit the flow of oxygen into the sensor 100, thereby improving the stability of sensors that are used in situations where variation in the partial pressure of oxygen causes non-linearity in sensor response. In these embodiments, the barrier layer 150 restricts oxygen transport by at least 40%, preferably at least 60%, and more preferably at least 80%, than the membrane restricts transport of the analyte. For a given type of polymer, films having a greater density (e.g., a density closer to that of the crystalline polymer) are preferred. Polyesters, such as polyethylene terephthalate, are typically less permeable to oxygen and are, therefore, preferred over polycarbonate membranes.

### C. Inserter

In one aspect of the invention, an inserter subassembly is provided. The inserter subassembly includes a rotor engaged to a shuttle that is coupled to an object to be inserted into a subject or user. A driver member is configured to translate the rotational motion of the rotor along a linear path, which includes the insertion path and retraction path.

The object to be inserted into the subject can be, for example, an analyte sensor as described above. Alternatively, other objects such as but not limited to an infusion set, or lancing device can be inserted.

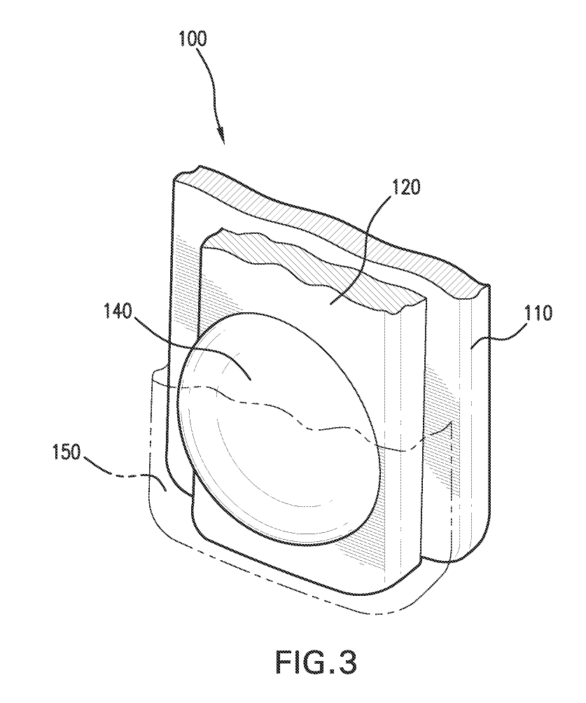

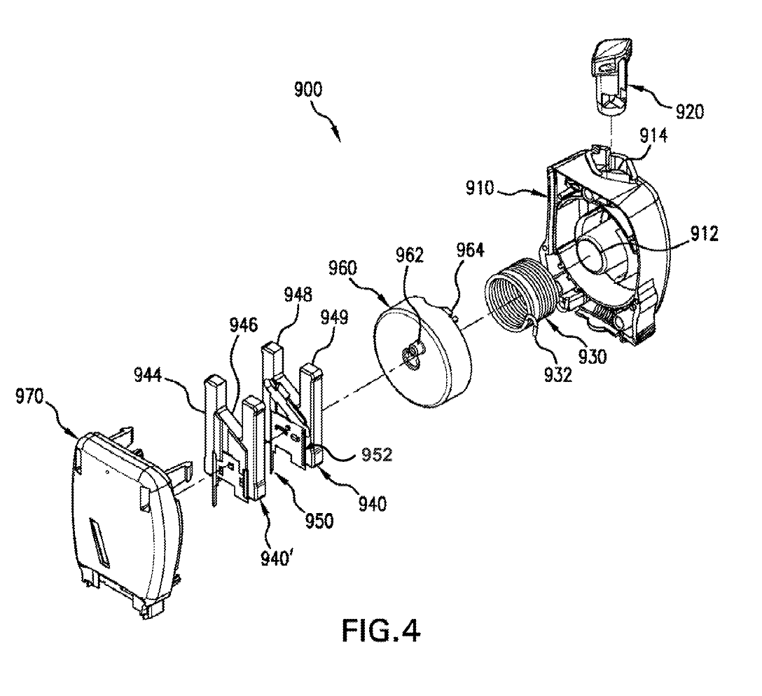

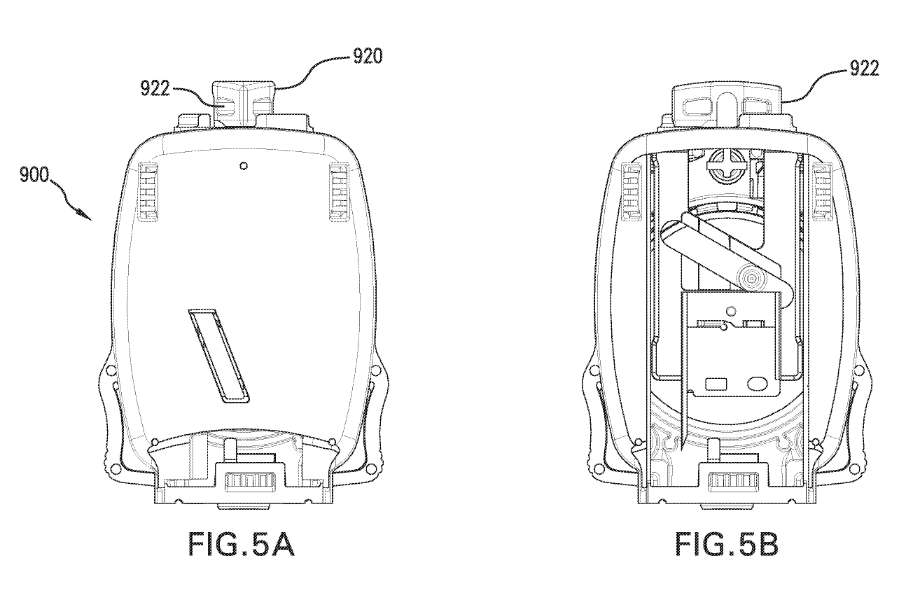

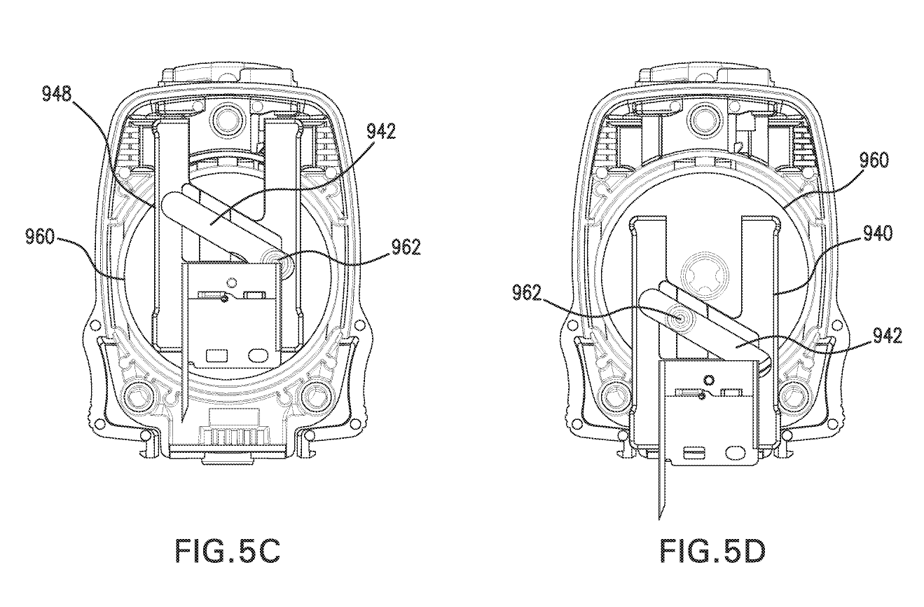

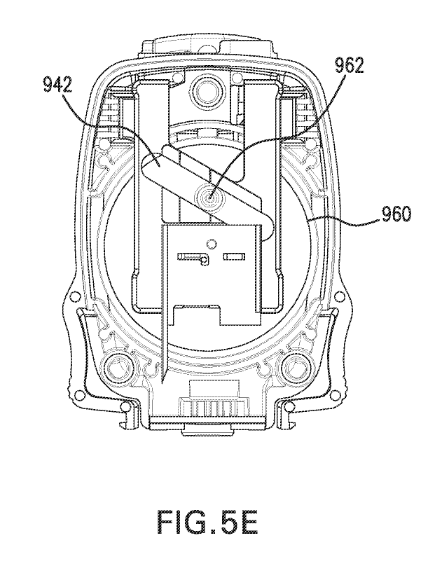

In one embodiment, as shown in FIG. 4, the inserter subassembly can be a component of an inserter assembly 900. The inserter subassembly includes rotor 960 and driver member 930. The drive member is configured to store and provide energy to drive the rotational movement of the rotor, which is then translated into linear movement of the shuttle. In this regard, the shuttle 940 is engaged to the rotor 960, such as by a slidingly engaged relationship, and/or non-rotatably engaged. For example, the rotor 960 can include a drive pin 962 configured to be received in an elongated channel or slot 942 (See, e.g., FIG. 5) formed in the second surface 946 of the shuttle 940. The shuttle 940 can be constrained in movement to a linear direction by guiding features, e.g., provided on the lid housing 970. Therefore, engagement of the rotor and shuttle can render the rotor capable of urging shuttle movement. In this manner, as the rotor moves along its rotational path, the shuttle coupled to the rotor, moves in a linear direction.

The linear path of the shuttle includes a reciprocating motion, e.g., an insertion direction, insertion point, refraction direction, and retraction point. Accordingly, at the insertion point of the linear path, the shuttle disassociates with the object to be inserted into the subject. For example, the sensor is released from the shuttle. In some embodiments, the sensor is retained with a frictional fit in the shuttle. When the sensor is inserted into the skin of a subject, the sensor may overcome the frictional fit in which the sensor is retained by the shuttle. For example, the sensor may include a barb or bead or other retention member which engages the skin of the subject. In some embodiments, a mounting unit may be provided which includes an engagement member which engages the sensor, e.g., a aperture and bead disposed on the sensor and mounting unit. Further details regarding the sensor and shuttle are discussed in U.S. Pat. No. 7,381,184, which is incorporated by reference herein for all purposes. The rotor continues to move along its rotational path. As the rotor continues its revolution along its rotational path and the shuttle continues an upward linear motion, for example, along the retraction direction, until the shuttle is retracted at the retraction point.

In one embodiment, shuttle 940 is formed from a generally planar substrate having opposing first and second surfaces 944, 946, and upwardly extending first and second arms 948, 949. An introducer sharp 950 can be mounted on a first surface 944 of the generally planar substrate, and an elongated channel 942 (as best seen in FIG. 5C) is formed in a second opposing surface 946 of the generally planar substrate. The elongated channel or slot can be configured to have a linear configuration, or alternatively an angled or curved configuration.

In one embodiment, the rotor 960 can include a hub arrangement disposed on the surface opposing the drive pin 962. The hub arrangement includes a tubular sleeve configured to receive or be received within the tubular sleeve or receptacle 912 of housing 910. A driver member 930 is disposed between housing 910 and rotor 960. In one embodiment as shown in FIG. 4, the driver member 930 can be a torsion drive spring. As further depicted, the hub arrangement of rotor 960 can include a generally circular flange centrally disposed on the surface of the rotor body. The circular flange can be configured with a protrusion, such as for example, a spline, hex post, or square post, or other projection or protrusion, configured to engage the drive member, e.g., spring 930. The spring 930 can include a catch member 932, such as an arcuate shaped or generally U-shaped member, disposed at least one end of the spring 930. The catch member 932 is configured to engage the rotor protrusion 960. In this manner, the rotor 960 is capable of winding the spring 930 upon rotation of the rotor body and protrusion.

The resultant linear shuttle paths include, e.g., an insertion point and a retraction point. In this manner, as best viewed in FIGS. 5C to 5E, rotor 960 urges shuttle 940 in the insertion direction towards an insertion position (FIG. 5D) and subsequently towards a retraction position (FIG. 5E), by way of its engagement to shuttle during rotation of rotor 960.

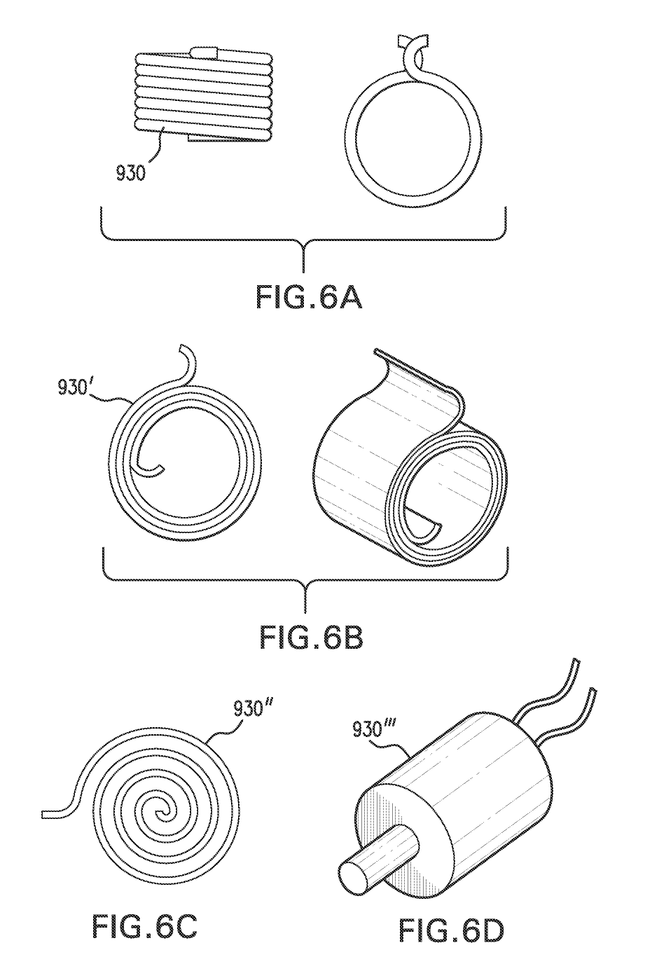

The driver member, for example, the spring 930, forces the rotor 960 along its rotational path to initiate the insertion of the object to be inserted into the subject. As illustrated in FIG. 4 and FIG. 6A, the driver member 930 of the inserter subassembly can be a torsion spring. Alternatively, in some embodiments, the driver member can take the form of various other types of springs, such as but not limited to constant force spring 930′ shown in FIG. 6B, or a spiral torsion spring 930″ shown in FIG. 6C, or motor 930′″ shown in FIG. 6D. The motor 930′ can be configured to urge the rotation of the rotor 960. Motor 930′″ can be operable to drive a shaft directly, or through a gear system, on which the rotor is fixed. The motor can be powered by an external power source. For example, actuation of the motor can drive the rotor through its rotation, which would then drive the shuttle though its linear movement, including the insertion direction, insertion point, retraction direction and retraction point.

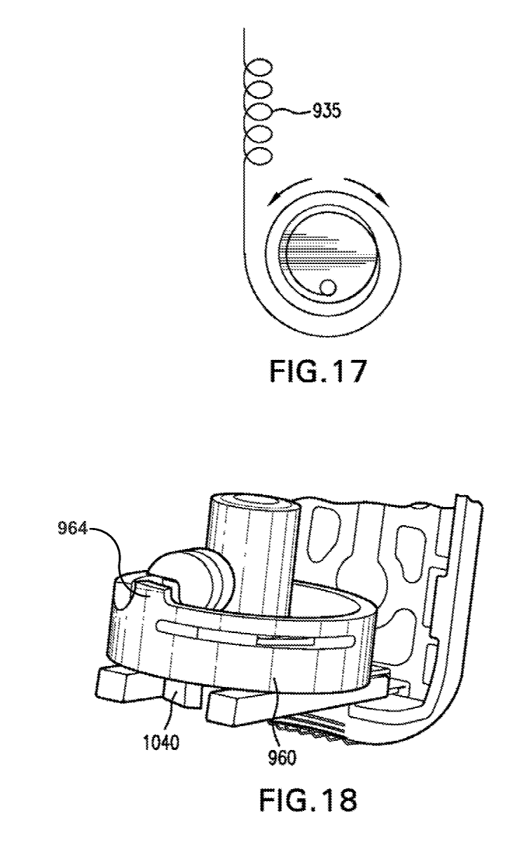

In another embodiment, the drive member 930 can include an elastic member 935 such as an elastic band, extension spring coupled to a flexible member, coil, or spiral member, as depicted in FIG. 17. In this regard, one end of the elastic member can be attached to the housing and the other end of the elastic member can be attached to the rotor. In the armed position, a flexible portion of the spring element can be wrapped around a feature having a profile disposed at a radial distance to the rotation axis of the rotor. For example, but not limitation, the profile can be a cylinder concentric to the axis of rotation to the rotor member. In this manner, as the spring element energy is released, the rotor is unwound. The elastic member can be positioned such that even as the rotor reaches its unwound position, there is residual tension on the member to provide active tension in maintaining the shuttle at its refracted point.

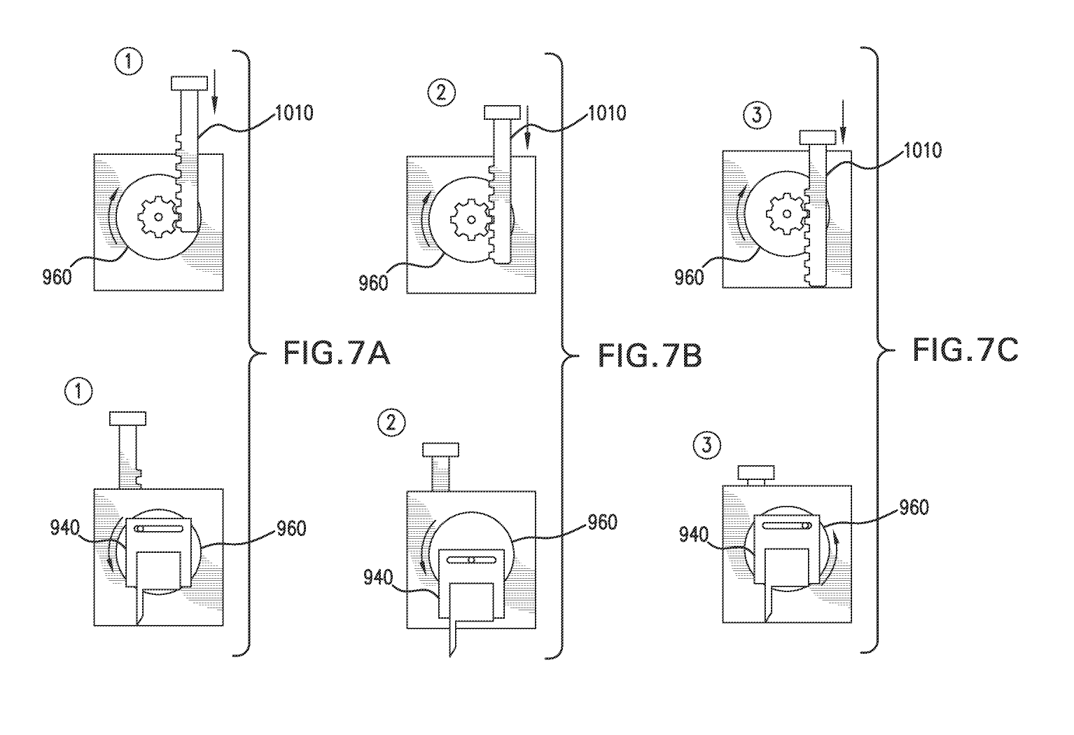

In yet another embodiment, the driver member engagement to the rotor can include a rack 1010, and the rotor 960 can include a pinion type mechanism, as illustrated in FIGS. 7A to 7C. As illustrated in FIG. 7A, the rotor includes a plurality of teeth configured to engage a plurality of grooves formed in a rack. In some embodiments, shuttle 940 is provided with a slot 942 which is engaged by a drive pin 962 on rotor 960. The linear movement of the rack can drive the rotational motion of the rotor, which can then urge the shuttle 940 along the linear path down the rack and toward the insertion direction, as shown in FIG. 7B. As illustrated in FIG. 7C, as the rack continues its downward movement, the rotor continues its revolution, and the shuttle is urged upward toward the retraction position. As illustrated in FIGS. 7A to 7C, the rotor and shuttle path includes a pre-insertion position (7A), insertion position (7B), and retracted position (7C).

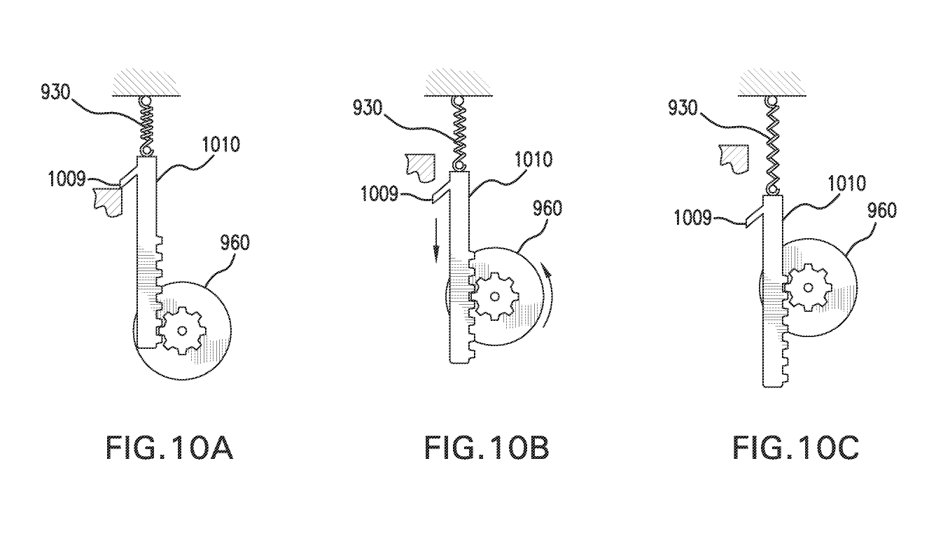

In some embodiments, the rack can further include a spring member, such as an extension spring, as shown in FIGS. 9A-9C or a compression spring as shown in FIGS. 10A-10C. Other springs, however, can be utilized. In this manner, as illustrated in FIGS. 9A-9C, energy is stored in spring 930. When an actuator, or trigger 1009, is released, the stored energy on the extension spring 930 is released, the rack 1010 is driven downward, and the rotor 960 rotates along its rotational path. Thus, the shuttle 940 and sharp 950 are driven through insertion and then retraction. Likewise, as illustrated in FIGS. 10A-10C, the spring 930 can be initially compressed. As the compressed energy is released, the rack 1010 can be driven downward and as the rotor 960 rotates along its rotational path as illustrated in FIGS. 10A and 10B, the shuttle 940 (not shown in FIGS. 10A-10C) is urged toward the insertion and then retraction positions.

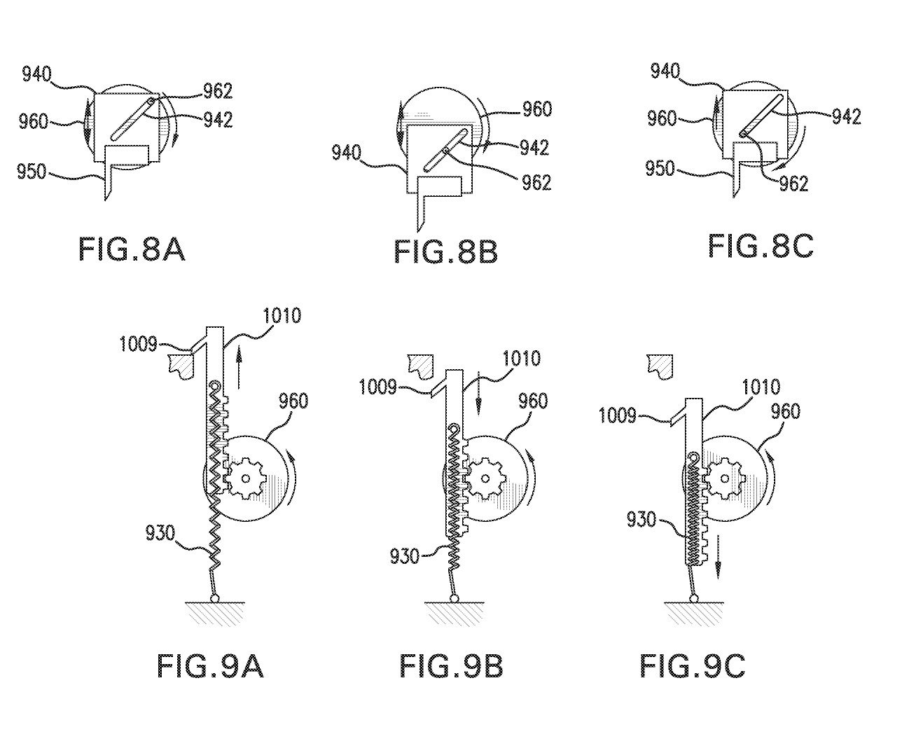

Additionally, as illustrated in FIG. 8A to 8C, which depicts the front view of an inserter subassembly including the rack and pinion with spring, the rotation of the pinion drives the shuttle 940 in a downward motion beginning with a position illustrated in FIG. 8A towards an insertion position illustrated in FIG. 8B, and then upward towards a retraction position illustrated in FIG. 8C, as the extension spring of FIGS. 9A to 9C extends and retracts, and as compression spring of FIGS. 10A to 10C compresses and is ultimately released.

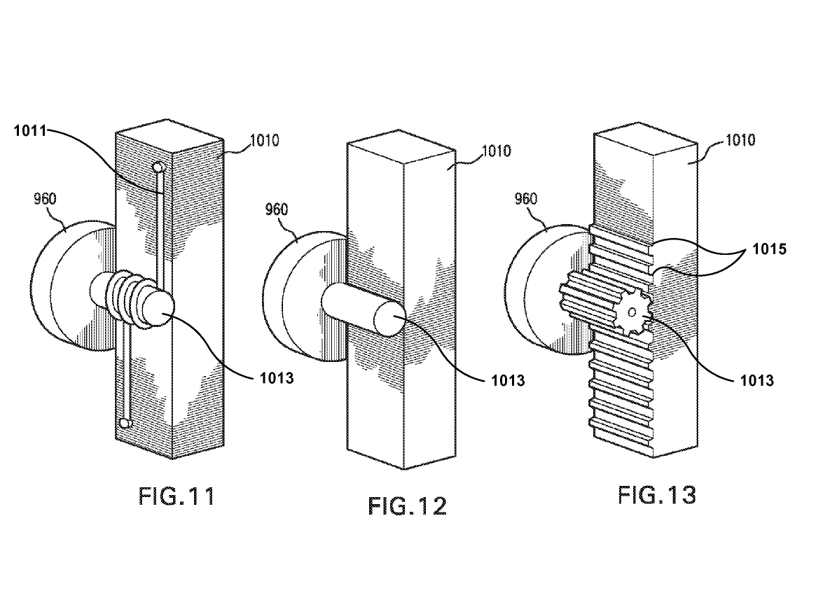

In yet another embodiment, as an alternative to the toothed engagement, the rack and pinion can include a cable 1011, as depicted in FIG. 11. In this embodiment, a cable is disposed along the rack 1010. At least a portion of the cable 1011 is disposed about a pinion 1013 disposed on the rotor. As the rack is moved up or down, the motion can be translated to rotation of the pinion 1013. In another embodiment, the rack 1010 and pinion 1013 include a frictional engagement, as illustrated in FIG. 12. The friction engagement can act equivalently to the toothed engagement, such that only friction between the pinion 1013 and rack 1010 surfaces provides the engagement between the two surfaces.

In yet another embodiment, the rack 1010 and pinion 1013 include a plurality of corresponding teeth and grooves for engagement, as illustrated in FIG. 13. For example, the rack 1010 includes a plurality of teeth 1015, and the pinion is a gear including a plurality of grooves 1017. The rack 1010 and pinion 1013 converts the linear movement of the rack 1010 to the rotation of the rotor 960, which then drives the linear movement of the shuttle (not shown). In this manner, the diameter of the pinion 1013 can be configured to determine the speed that the shuttle moves as the pinion rotation is dependent on the linear motion of the rack and the pitch diameter of the pinion.

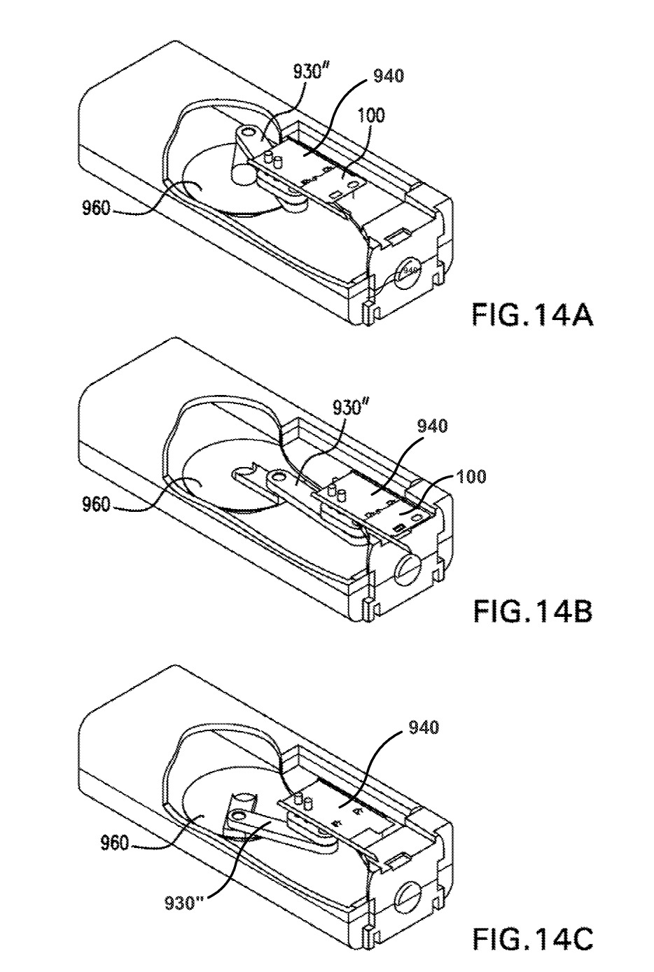

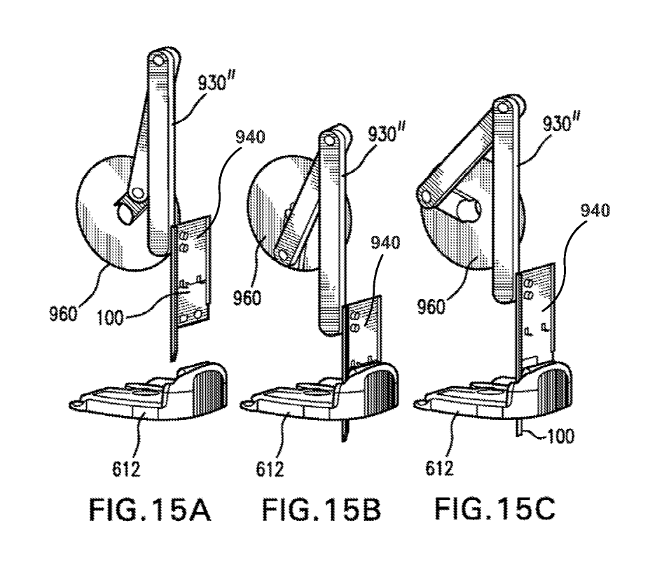

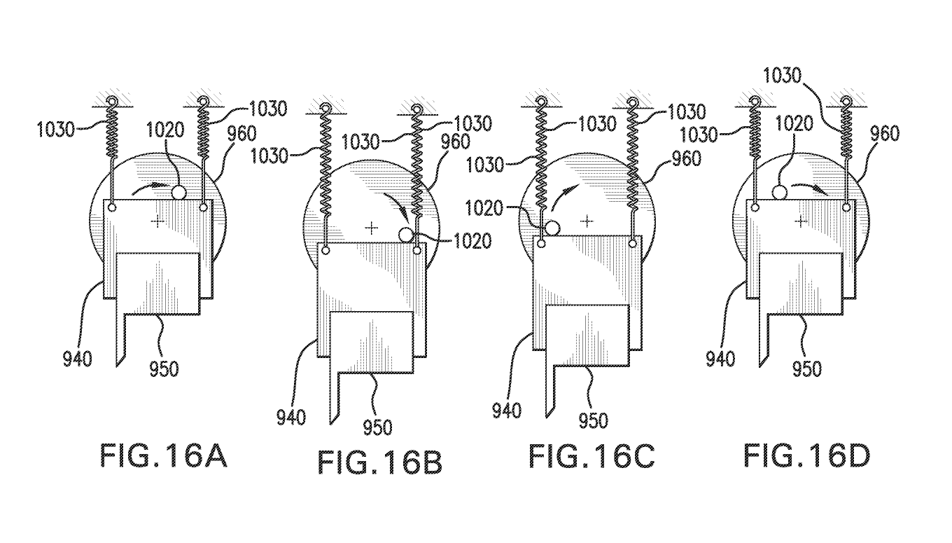

In another embodiment, a linkage, which has one end coupled to the rotor and a second end coupled to the shuttle provides an alternate method of translating the rotary movement of the rotor to the linear movement of the shuttle. The linkage 930″can be configured to translate rotational movement of the rotor 960 to linear motion of the shuttle, as shown in FIGS. 14A, 14B, and 14C. In this manner, rotation of the rotor 960 translates to linear movement of the shuttle 950 (which may or may not be integral with the sharp), which is urged toward an insertion direction and back towards a retraction direction, as illustrated in FIGS. 14A to 14C. As shown, the rotor 960 rotates through a portion of the full 360° rotation, thereby causes linear motion of shuttle 940, as shown in FIGS. 14A to 14C and 15A to 15C.

In yet another embodiment, the rotor can include a cam 1020, e.g., a projecting part of the rotating rotor 960, which strikes and urges the shuttle 940 downward towards an insertion direction, as illustrated in FIGS. 16A-16D. The subassembly can further include a one or more springs 1030 secured to the shuttle 940, such as, but not limited to an extension spring. The spring is configured to provide an upward force of the shuttle in the retraction direction to the refraction point, as illustrated in FIGS. 16A-16D. As illustrated, for this embodiment, the upward movement of the shuttle can be limited by the rotor cam motion. For example, the cam can be configured to constrain the upward motion of the spring.

In some embodiments, referring back to FIG. 4, the inserter subassembly is part of a sensor inserter assembly, which includes housing 910, actuator button 920, and lid 970. Sensor (not shown in FIG. 4) has a main surface slidingly mounted between U-shaped rails or flanges 952 of introducer sharp 950 and is releasably retained on the introducer sharp by a dimple or protrusion extending laterally from the main surface of the sensor body, and which engages introducer flange 952. Introducer sharp 950 is mounted to a surface of the shuttle 940, for example by snap-on fit, interference fit, adhesive, heat stake or ultrasonic weld.

The lid 970 can be configured to include guides to maintain linear movement of the shuttle and inhibit rotational movement of the shuttle.

As shown in FIG. 5, actuator button 920 can include an actuator trigger pin that is slidingly received within and resides in an aperture 914 shown in FIG. 4, disposed at a proximal end of housing 910. In one embodiment, as shown in FIGS. 5A and 5B, actuator button 920 is configured to include a plurality of relative positions to housing 910 and aperture 914. For example, actuator button can be disposed in a safety position (FIG. 5A), pre-insertion position (FIG. 5B), and insertion position (FIG. 5C). While in the safety position, actuator button 920 is limited to no longitudinal movement. Longitudinal movement of the actuator button is prevented by the proximal head 922 of actuator button 920 contacting the edge of the aperture 914 of housing 910. Thus, the single actuator, for example, a trigger pin, provides both a safety position, a safety-removed or pre-fire position, and positive actuation.

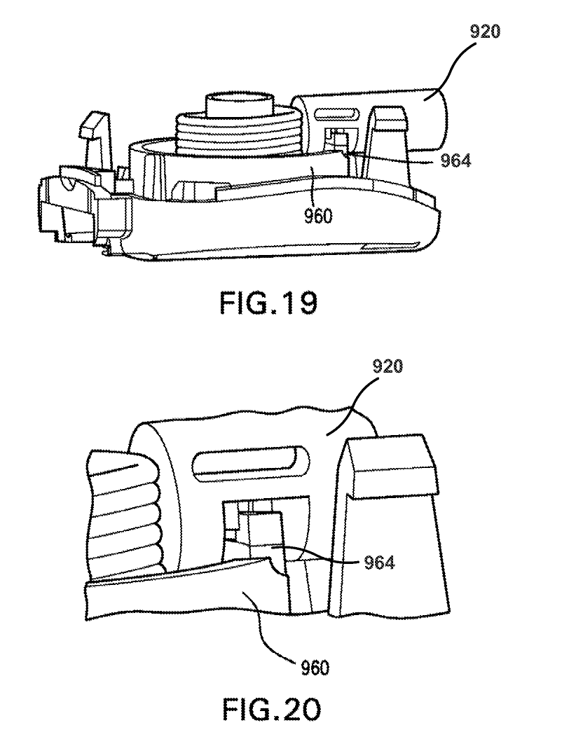

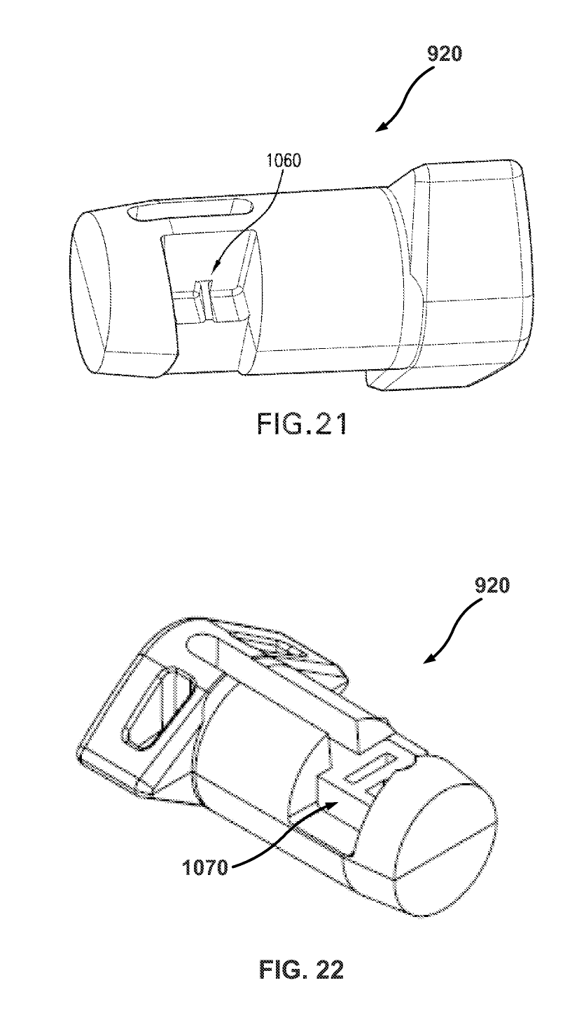

During operation, actuator button 920 is rotated (e.g., about ¼ of a turn) to enter the pre-fire position, as shown in FIG. 5B. While in the pre-insertion (pre-fire) position (FIG. 5C), the actuator button is capable of being depressed, thereby releasing the rotor 960. Upon release of the rotor 960, the rotor 960 initiates a revolution along the rotational path, which is urges the shuttle along a linear path towards the insertion direction, as described above. After release of the sensor from the shuttle 940, the rotor 960 continues along the rotational path thereby urging the shuttle toward a retraction position (FIG. 5E). In this aspect, rotor 960 can include a stop 964 configured to halt rotation upon frictional interference with the trigger pin. In other embodiments, the rotor 960 can be configured to include a brake 1040, as illustrated in FIG. 18. In this manner, the brake 1040 can be a flexible member on the rotor to act as a friction brake against to dissipate excess energy and to slow down or stop the rotor from rotation. For example, the brake is configured to engage at least one of first and second arms 948, 949 of shuttle 940 to slow down or stop rotation. In another embodiment, as illustrated in FIGS. 19 and 20, the actuator 920 can be configured with one or more ribs such as a rib, or a button 1060 as illustrated in FIG. 21 to engage the stop 964 on the rotor such that the rotor is slowed down or rotation is stopped by the interference with the one or more ribs after the rotor has made at least a partial rotation. In some embodiments, the rib is a protrusion capable of deforming upon contact with the stop 964, e.g., a crush rib. In some embodiments, the rib does not deform upon contact with the stop 964. According to a further embodiment as illustrated in FIG. 22, the actuator 920 is provided with a dampening member 1070 which is included within actuator 920. Dampening member 1070 is fabricated from a material having a lower durometer than the body of the actuator 920. For example, the dampening member 1070 may be fabricated from an elastomeric material. The dampening member 1070 engages the stop 964 disposed on the rotor and provides sound dampening for the engagement between the brake and the actuator.

The stopped rotor remains against the actuator to keep the sharp safety inside the housing. In this manner, the stop 964 projecting from the rotor surface contacts the actuator button at the completion of the desired rotation of the rotor. For example, the rotor is permitted to rotate such that shuttle 940 traverses a reciprocal linear path between an initial retracted position, and insertion position, and back towards the retracted position before the stop 964 engages the rib 1060 or dampening member 1070. Near the conclusion of the rotor rotation, shuttle (with downwardly projecting sharp) is retracted upwardly into the housing 910 to a retracted position, as shown in FIG. 5E. To this end, the revolution of the rotor (with drive pin engaged within the elongated channel of shuttle) positions the shuttle for the fire, insert, and retracted positions. Thus, the rotor pin controls the movement during insertion and extraction of the sharp.

In general, the inserter assembly can be constructed to include only five molded parts, one spring, and one sharp. Accordingly, the inserter assembly of the invention has the added benefit of reduced manufacturing costs. The ease of assembly is designed for automated process with bottom up assembly. In one embodiment, standard ABS material and standard tolerances are utilized.

As described, in accordance with one embodiment of the invention, insertion of the sensor 100 into the user's body is facilitated by an inserter assembly. Generally, the inserter assembly can be preloaded with the sensor.

### D. Mount

As described above, the inserter assembly can be configured to couple to a mount. For example, in some embodiments, the sensor 100 is configured to be an on-body unit that is at least partially placed on or below the skin of a user. As schematically depicted in FIG. 15A, sensor is positioned on a user's skin by a mounting device 612. In some embodiments, the mounting device 612 includes a receptacle or slot (not shown) to receive a sensor and can be attached to the user's skin by a variety of techniques including, for example, adhering directly onto the skin with an adhesive provided on at least a portion of the mounting unit, such that the adhesive is the sole source of adhesion. In one embodiment, the inserter assembly can be part of an insertion kit, which includes the inserter assembly described above, the sensor, and a mounting unit.

In one embodiment, the mount 612 is formed as a single integral component. However, other embodiments, include a modular mount device in which the separate components are integrally connected to form a unitary component. After deployment of the sensor into the user's body, a transmitter is engaged with both the mount and the sensor. In this regard, the electronic circuitry of the transmitter makes electrical contact with contacts on the sensor while transmitter is seated in mount 612. Sensor, mount 612, and transmitter remain in place on the user's body for a predetermined period, e.g., three to seven days. These components are then removed so that sensor and mount can be properly discarded and replacement components can be utilized. The mount assembly including the sensor and transmitter usually includes no wires, catheters, or cables to other components.

Additional detailed description of embodiments of the disclosed subject matter are provided in but not limited to: U.S. Pat. No. 7,299,082; U.S. Pat. No. 7,167,818; U.S. Pat. No. 7,041,468; U.S. Pat. No. 6,942,518; U.S. Pat. No. 6,893,545; U.S. Pat. No. 6,881,551; U.S. Pat. No. 6,773,671; U.S. Pat. No. 6,764,581; U.S. Pat. No. 6,749,740; U.S. Pat. No. 6,746,582; U.S. Pat. No. 6,736,957; U.S. Pat. No. 6,730,200; U.S. Pat. No. 6,676,816; U.S. Pat. No. 6,618,934; U.S. Pat. No. 6,616,819; U.S. Pat. No. 6,600,997; U.S. Pat. No. 6,592,745; U.S. Pat. No. 6,591,125; U.S. Pat. No. 6,560,471; U.S. Pat. No. 6,540,891; U.S. Pat. No. 6,514,718; U.S. Pat. No. 6,514,460; U.S. Pat. No. 6,503,381; U.S. Pat. No. 6,461,496; U.S. Pat. No. 6,377,894; U.S. Pat. No. 6,338,790; U.S. Pat. No. 6,299,757; U.S. Pat. No. 6,299,757; U.S. Pat. No. 6,284,478; U.S. Pat. No. 6,270,455; U.S. Pat. No. 6,175,752; U.S. Pat. No. 6,161,095; U.S. Pat. No. 6,144,837; U.S. Pat. No. 6,143,164; U.S. Pat. No. 6,121,009; U.S. Pat. No. 6,120,676; U.S. Pat. No. 6,071,391; U.S. Pat. No. 5,918,603; U.S. Pat. No. 5,899,855; U.S. Pat. No. 5,822,715; U.S. Pat. No. 5,820,551; U.S. Pat. No. 5,628,890; U.S. Pat. No. 5,601,435; U.S. Pat. No. 5,593,852; U.S. Pat. No. 5,509,410; U.S. Pat. No. 5,320,715; U.S. Pat. No. 5,264,014; U.S. Pat. No. 5,262,305; U.S. Pat. No. 5,262,035; U.S. Pat. No. 4,711,245; U.S. Pat. No. 4,545,382; U.S. patent application Ser. No. 10/745,878 filed Dec. 26, 2003, U.S. patent application Ser. No. 12/698,129, filed Feb. 1, 2010; U.S. Patent Application No. 61/317,243, filed Mar. 24, 2010, U.S. Patent Application No. 61/345,562, filed May 17, 2010; U.S. Patent Application No. 61/249,535, filed Oct. 7, 2009, U.S. Patent Application No. 61/246,825, filed Sep. 29, 2009, U.S. Patent Application No. 61/361,374, filed Jul. 2, 2010, the disclosures of each of which is incorporated herein by reference herein for all purposes.

What is claimed is:

1. An inserter subassembly for insertion of a medical device into the skin of a subject, comprising:
a rotor capable of moving along a rotational path;
a driver member engaged to the rotor, wherein the driver is capable of urging the rotor along the rotational path;
a shuttle configured to receive a medical device to be inserted into a subject, the shuttle being coupled to the rotor,
wherein the shuttle is urged along a reciprocal linear path comprising an insertion direction and a retraction direction, as the rotor moves along the rotational path.

2. The inserter assembly of claim 1, wherein the medical device to be inserted into a subject comprises an analyte sensor, infusion set, or lancing device.

3. The inserter assembly of claim 1, wherein the rotor is a disc shaped member.

4. The inserter subassembly of claim 1, wherein the rotor comprises a pin to engage the shuttle.

5. The inserter subassembly of claim 4, wherein the shuttle comprises a channel to receive the rotor pin.

6. The inserter subassembly of claim 5, wherein the channel has a linear, non-linear, curved, angled, horizontal, pocket, slot, or pocket configuration.

7. The inserter subassembly of claim 6, wherein the pin and channel engagement translates a single direction rotation of the rotor to the reciprocal linear path toward the insertion direction and the retraction direction.

8. The inserter subassembly of claim 1, wherein the driver member is a spring.

9. The inserter subassembly of claim 8, wherein the spring comprises a torsion drive spring, a constant force spring, or a spiral torsion spring.

10. The inserter subassembly of claim 1, wherein the driver member comprises a rack, and further wherein the rotor comprises a pinion.

11. The inserter subassembly of claim 1, wherein the driver member comprises a linkage member having a first end secured to the rotor and a second end secured to the shuttle.

12. The inserter subassembly of claim 11, wherein the linkage member translates the rotational path of the rotor to a linear path of the shuttle.

13. The inserter subassembly of claim 1, wherein the rotor engages a cam disposed on the shuttle to urge the shuttle downward towards an insertion position.

14. The inserter subassembly of claim 13, wherein a spring member urges the shuttle upward towards a retraction position.

15. An inserter assembly for an analyte monitoring system, the inserter assembly comprising:
a housing;
a rotor capable of moving along a rotational path;
a driver member engaged to the rotor, wherein the driver is capable of urging the rotor along the rotational path;
a shuttle configured to receive an object to be inserted into a subject, the shuttle being coupled to the rotor and movably connected to the housing, wherein the shuttle is urged along a linear reciprocal path toward an insertion direction and a retraction direction as the rotor moves along the rotational path,
an introducer sharp coupled to the shuttle, the introducer sharp configured to releasably receive a sensor; and
a sensor releasably coupled to the introducer sharp.

16. The inserter assembly of claim 15, wherein the housing defines a guide to maintain the shuttle along the linear path.

17. The inserter assembly of claim 16, wherein the housing further comprising a lid portion defining the guide.

18. The inserter assembly of claim 15, wherein the rotor is a disc shaped member.

19. The inserter assembly of claim 15, wherein the rotor has a non-circular shape.

20. The inserter assembly of claim 15, wherein the rotor comprises a pin to engage the shuttle.

21. The inserter assembly of claim 20, wherein the shuttle comprises an elongated channel to receive the rotor pin.

22. The inserter assembly of claim 21, wherein the elongated channel has a linear configuration.

23. The inserter assembly of claim 21, wherein the elongated channel has a curved configuration.

24. The inserter assembly of claim 21, wherein the pin and channel engagement translates a single direction rotation of the rotor to the reciprocal linear path toward the insertion and refraction direction.

25. The inserter assembly of claim 15, wherein the driver member is a spring.

26. The inserter assembly of claim 25, wherein the spring is a torsion drive spring, a constant force spring, or a spiral torsion spring.

27. The inserter assembly of claim 25, wherein the driver member comprises a rack and the rotor comprises a pinion.

28. The inserter assembly of claim 15, wherein the driver member comprises a linkage member having a first end secured to the rotor and a second end secured to the shuttle.

29. The inserter assembly of claim 15, wherein the rotor engages a cam disposed on the shuttle to urge the shuttle downward towards an insertion position.

30. The inserter assembly of claim 29, further comprising a spring member to urge the shuttle upward towards a retraction position.

31. The inserter assembly of claim 15, wherein the rotor comprises a protrusion configured to engage at least one of the housing or the shuttle to slow down rotation of the rotor.

32. The inserter assembly of claim 15, further including an actuator to actuate the driver member such that the shuttle is urged towards the insertion direction.

33. The inserter assembly of claim 32, wherein the actuator is configured to release the driver member from a compressed configuration towards an expanded configuration.

34. The inserter assembly of claim 32, wherein the actuator comprises a safety to impede the actuator until the safety is deactivated.

35. The inserter assembly of claim 32, wherein the actuator comprises an engagement member configured to engage the rotor.

36. The inserter assembly of claim 35, wherein the engagement between the actuator engagement member and the rotor inhibits rotational motion of the rotor.

37. The inserter assembly of claim 36, wherein the engagement member engages the rotor following a predetermined rotation of the rotor.

38. The inserter assembly of claim 36, wherein the rotor comprises a protrusion configured to engage the engagement member.

39. The inserter assembly of claim 35, wherein the engagement member is a crush rib.

40. The inserter assembly of claim 35, wherein the engagement member is fabricated from a material having a lower durometer than the actuator.

41. The inserter assembly of claim 35, wherein the engagement member is fabricated from an elastomeric material.

42. The inserter assembly of claim 15, wherein the sensor is released from the inserter assembly at an insertion site.

43. The inserter assembly of claim 15, wherein the sensor is at least partially implanted in a subject at an insertion site.

44. The inserter assembly of claim 15, further comprising a mounting unit adapted to attach to a subject's body at an insertion site.

45. The inserter assembly of claim 44, wherein the mounting unit is adapted to position the sensor with respect to the subject's skin.
